(19) United States
(12) Patent Application Publication
(10) Pub. No.: US 2011/0020884 A1
(43) Pub. Date: Jan. 27, 2011

(54) METHOD FOR THE CONVERSION OF PLANT MATERIALS INTO FUELS AND CHEMICALS BY SEQUENTIAL ACTION OF TWO MICROORGANISMS

(76) Inventors: William Greg Latouf, Ashland, MA (US); John Kilbane, Houston, TX (US)

Correspondence Address: WILSON, SONSINI, GOODRICH & ROSATI, 650 PAGE MILL ROAD, PALO ALTO, CA, 94304-1050, US

(21) Appl. No.: 12/919,750

(22) PCT Filed: Feb. 27, 2009
(86) PCT No.: PCT/US2009/035597

§ 371 Date: Oct. 12, 2010

Related U.S. Application Data
(60) Provisional application No. 61/032,048, filed on Feb. 27, 2008.

(51) Int. Cl. C12P 7/40; C12P 7/24; C12P 7/26; C12P 7/02; C12P 7/14

(52) U.S. Cl. 435/136; 435/147; 435/148; 435/155; 435/162

(57) ABSTRACT

A process is disclosed for converting complex plant polysaccharides, including cellulosic materials, into fuels and other chemicals. In preferred embodiments, the process comprises sequential hydrolysis of the plant polysaccharides by two or more microorganisms

### CROSS-REFERENCE

This application claims the benefit of U.S. Provisional Application No. 61/032,048 filed on Feb. 27, 2008, which application is incorporated herein by reference.

### BACKGROUND OF THE INVENTION

There is an interest in developing methods of producing usable energy from renewable and sustainable biomass resources. Energy in the form of carbohydrates can be found in waste biomass, and in dedicated energy crops, such as grains (e.g., corn or wheat) or grasses (e.g., switchgrass). Cellulosic and lignocellulosic materials, are produced, processed, and used in large quantities in a number of applications.

A current challenge is to develop viable and economical strategies for the conversion of carbohydrates into usable energy forms. Strategies for deriving useful energy from carbohydrates include the production of ethanol (“cellulosic ethanol”) and other alcohols (e.g., butanol), conversion of carbohydrates into hydrogen, and direct conversion of carbohydrates into electrical energy through fuel cells. For example, biomass ethanol strategies are described by DiPardo, Journal of Outlook for Biomass Ethanol Production and Demand (EIA Forecasts), 2002; Sheehan, Biotechnology Progress, 15:8179, 1999; Martin, Enzyme Microbes Technology, 31:274, 2002; Greer, BioCycle, 61-65, April 2005; Lynd, Microbiology and Molecular Biology Reviews, 66:3, 506-577, 2002; and Lynd et al. in “Consolidated Bioprocessing of Cellulosic Biomass: An Update,” Current Opinion in Biotechnology, 16:577-583, 2005.

While the production of ethanol from grains and sugars, are well known, such methods involve diverting valuable food crops into energy production. Accordingly, it would be desirable to less costly and more abundant plant materials, such as lignocellulosic materials, into ethanol. Unfortunately, the efficient and complete bioconversion of plant-derived polymers that comprise lignocellulosic material (cellulose, hemicelluloses, and lignin) to fuels and/or chemicals while highly desirable, is challenging because only the cellulose and hemicelluloses components of lignocellulosic material can be converted by currently known biocatalysts into fuels such as ethanol. Moreover, the compact/dense structure of the material, combined with the indigestibility of the lignin component of lignocellulosic material, limits the bioavailability of substantial portions of cellulose and hemicelluloses in lignocellulosic material. Not all organisms can utilize these complex compounds and structures. Frequently, organisms that can produce desirable end products, including compounds which can be utilized as fuels or as other functional compounds, prefer simple sugars or other particular carbon substrates.

Current processes for the bioconversion of lignocellulosic material to fuels and/or chemicals can include extensive and costly pretreatment of the material by mechanical, thermochemical, and biochemical processes. Generally, the goals of such pretreatment processes include (1) rendering the cellulosic and hemicellulosic polymers more accessible to microorganisms, and (2) converting the complex cellulosic and hemicellulosic polysaccharides into simpler, fermentable sugars or other simple compounds, that are more readily converted into fuels and other chemicals by microorganisms. The mechanical, thermochemical, and biochemical processes frequently used in the pretreatment of lignocellulosic material constitute a major cost and are not completely effective. Furthermore, the microorganisms currently used for the production of fuels and other chemicals from lignocellulosic material lack the necessary cellular machinery for both breaking down the complex plant polysaccharides into sugars (saccharification) and then converting the various resulting sugars into fuels and other chemical products in an efficient manner. Thus, there remains a substantial unmet need for bioconversion processes that take advantage of more versatile microorganisms and/or combinations of microorganisms in order to saccharify complex polysaccharides and more efficiently convert a broader spectrum of fermentable sugars into fuels and other chemicals.

### SUMMARY OF THE INVENTION

The invention provides a process for converting complex plant polysaccharides, including cellulosic materials, into fuels and other chemicals. In preferred embodiments, the process comprises conversion of plant polysaccharides into shorter chain polysaccharides or other compounds by one organism which are then used as a substrate by another organism for the production of the desired compounds. In other preferred embodiments, the process comprises sequential hydrolysis of the plant polysaccharides by a cellulolytic aerobic microorganism and fermentation of the hydrolysate by an anaerobic microorganism.

In a first aspect, a process is disclosed for producing a biofuel such as ethanol and other chemicals. The process comprises: (1) providing a biomass material under anaerobic conditions, where the biomass has not been treated with exogenously supplied chemicals or enzymes; (2) treating the biomass with a first culture of a non-genetically modified anaerobic bacterium, where the non-genetically modified anaerobic bacterium converts at least a portion of the biomass into monosaccharides and disaccharides; and (3) treating the biomass with a second culture of a microorganism that is not an obligate aerobe, where the monosaccharides and disaccharides are converted to a biofuel. In some embodiments this process takes place in a closed container. The first culture of non-genetically modified anaerobic bacterium can be Clostridium phytofermentans.

In a second aspect, a process is disclosed in accordance with a preferred embodiment of the present invention for making ethanol and other chemicals. The process comprises: (1) providing a pretreated biomass-derived material comprising a plant polysaccharide; (2) inoculating the pretreated biomass-derived material with a first culture comprising a cellulolytic aerobic microorganism in the presence of oxygen to generate an aerobic broth, wherein the aerobic microorganism is capable of at least partially hydrolyzing the plant polysaccharide; (3) incubating the aerobic broth until the cellulolytic aerobic microorganism consumes at least a portion of the oxygen and hydrolyzes at least a portion of the plant polysaccharide, thereby converting the aerobic broth into an anaerobic broth comprising a hydrolysate comprising fermentable sugars; (4) inoculating the anaerobic broth with a second culture comprising an anaerobic microorganism capable of converting the fermentable sugars into ethanol; and (5) fermenting the inoculated anaerobic broth until a portion of the fermentable sugars have been converted into ethanol.

In a third aspect, a three stage process is disclosed for producing a purified biofuel such as ethanol and other chemicals. The process comprises: (1) a first stage wherein a biomass material is treated under anaerobic conditions with a first culture of a non-genetically modified anaerobic bacterium, where the non-genetically modified anaerobic bacterium substantially converts the biomass into monosaccharides and disaccharides without exogenously supplied chemicals or enzymes; (2) a second stage where the monosaccharides and disaccharides are treated with a culture of a second microorganism, where the monosaccharides and disaccharides are converted to a biofuel; and (3) a third stage wherein the resulting biofuel is separated and recovered from the residual biomass and cultures. In some embodiments this process takes place in a closed container. The first culture of non-genetically modified anaerobic bacterium can be Clostridium phytofermentans.

In some embodiments of the above-described processes, at least a portion of the ethanol is recovered from the fermented anaerobic broth.

In other embodiments, the process further comprises a step of lysing the aerobic and anaerobic microorganisms in the fermented anaerobic broth to produce a lysate comprising remaining fermentable sugars and cellular contents. In a variation, the lysate may be subjected to additional physical and/or chemical treatment. In another variation, the lysate may be inoculated with another anaerobic microorganism capable of accelerating the conversion of the remaining fermentable sugars into ethanol and other chemicals.

In another aspect, a process is disclosed in accordance with a preferred embodiment of the present invention for making ethanol and other chemicals. The process comprises (1) providing a biomass-derived material comprising a plant polysaccharide; (2) inoculating the biomass-derived material with a first culture comprising cells of Clostridium phytofermentans under anaerobic conditions to hydrolyze at least a portion of the plant polysaccharide wherein the cells of the culture incorporate at least a portion of the hydrolyzed plant polysaccharide as an intracellular compound; (3) lysing the cells of Clostridium phytofermentans to produce a lysed broth; (4) inoculating the lysed broth with a second culture comprising an anaerobic microorganism capable of converting fermentable sugars into ethanol and other chemicals; and (5) fermenting the inoculated anaerobic broth until at least a portion of the fermentable sugars have been converted into ethanol and/or other chemicals.

In another aspect, a process is disclosed for producing a biofuel such as ethanol and other chemicals. The process comprises subjecting biomass which includes cellulose and hemi-cellulose containing plant materials to fermentation under mesophilic conditions in the presence of co-cultures of Clostridium phytofermentans and a second Clostridium species selected from the group consisting of Clostridium acetobutyliticum, Clostridium thermocellum, and Clostridium cellovorans, the ratio of the cultures being in an amount whereby the conversion ratios of cellulose:ethanol and hemi-cellulose:ethanol are greater than the ratios obtained by use of either Clostridium phytofermentans or the second Clostridium species alone.

In another aspect, a process is disclosed for producing a biofuel such as ethanol and other chemicals. The process comprises subjecting biomass which includes cellulose and hemi-cellulose containing plant materials to fermentation under mesophilic conditions in the presence of co-cultures of Clostridium phytofermentans and Zymonomas mobilis, the ratio of the cultures being in an amount whereby the conversion ratios of cellulose:ethanol and hemi-cellulose:ethanol are greater than the ratios obtained by use of either Clostridium phytofermentans or Zymonomas mobilis alone.

In another aspect, a process is disclosed for simultaneous saccharification and fermentation of cellulosic solids from biomass into biofuel such as ethanol or other chemicals. The process comprises treating the biomass in a closed container with a Clostridium phytofermentans bacterium under conditions wherein the Clostridium phytofermentans bacterium produces saccharolytic enzymes sufficient to substantially convert the biomass into monosaccharides and disaccharides and introducing a culture of a second microorganism wherein the second organism is capable of substantially converting the monosaccharides and disaccharides into biofuel.

In another aspect, a process is disclosed for producing a biofuel from a lignin-containing biomass. The process comprises: (1) contacting the lignin-containing biomass with an aqueous alkaline solution at a concentration sufficient to hydrolyze at least a portion of the lignin-containing biomass; (2) neutralizing the treated biomass to a pH between 7 to 8; (3) treating the biomass in a closed container with a Clostridium phytofermentans bacterium under conditions where the Clostridium phytofermentans bacterium produces saccharolytic enzymes sufficient to substantially convert the treated biomass into monosaccharides and disaccharides; and (4) introducing a culture of a second microorganism where the second organism is capable of substantially converting the monosaccharides and disaccharides into biofuel.

In another aspect, a process is disclosed for producing a biofuel and nutrient fermentation residual from biomass. The process comprises: (1) treating biomass with a culture comprising Clostridium phytofermentans that, in a fermentation reaction, produces an alcohol and a fermentation residual comprising a nutrient selected from the group consisting of amino acids, cofactors, hormones, proteins, vitamins and lipids; (2) fermenting the culture under conditions suitable for production of the biofuel and under conditions suitable for production the nutrient; (3) separating the biofuel from the culture; and (3) recovering the fermentation residual comprising the nutrient.

In another aspect, a process is disclosed for producing ethanol from a cellulosic substrate. The process comprises: (1) providing within a reaction vessel a reaction mixture in the form of a slurry comprising cellulosic substrate rendered anaerobic, saccharolytic enzymes, a Clostridium phytofermentans bacterium and optionally a Zymomonas mobilis bacterium; (2) agitating the reaction mixture for a first selected time interval, where the reaction mixture is reacted under conditions sufficient to initiate and maintain a fermentation reaction; (3) ceasing agitation of the reaction mixture for a sufficient period of time to permit insoluble substrate of the reaction mixture to settle during a second selected time interval, thereby forming an ethanol containing effluent layer substantially free of suspended solids and a residual solids layer; (4) removing from the reaction vessel the ethanol-containing effluent upon expiration of the second selected time interval, and before any further agitation; (5) adding a second reaction mixture, comprising the components of the reaction mixture of step (1), to the reaction vessel which contains the residual solids; and (6) repeating steps (2) through (5) to maintain a continuous fermentation reaction.

In another aspect, the invention provides a composition comprising fermenting biomass containing a first culture of Zymomonas mobilis and a second culture of Clostridium phytofermentans in an amount not inhibiting Zymomonas growth and fermentation, in the substantial absence of exogenously supplied enzymes, air and added nutrients.

In another aspect, the invention provides a closed system process for the production of ethanol. The system comprises: (1) carrying out ethanol-producing anaerobic fermentation of biomass in a fermentation vessel at a temperature of at least about 35° C. in the presence of a Clostridium phytofermentans bacterium capable of producing sugars from biomass and in the presence of a facultatively anaerobic bacterium capable of fermenting sugars both aerobically and anaerobically and producing ethanol in anaerobic fermentation, (2) continuously withdrawing a portion of the fermentation medium from anaerobic fermentation; (3) separating bacteria from the withdrawn fermentation medium and recycling the separated bacteria to anaerobic fermentation; and (4) removing ethanol from the withdrawn portion of the fermentation medium.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 depicts a block diagram showing schematically the process of one embodiment of the invention.

FIG. 2 shows the growth of C. phytofermentans Stocks (0.3% CB.MB)

### DETAILED DESCRIPTION OF THE INVENTION

### Definitions

Unless defined otherwise, technical and scientific terms used herein have the same meaning as commonly understood by one of ordinary skill in the art to which this invention belongs. For purposes of the present invention, the following terms are defined below.

“Biofuels”, “Fuels and or other chemicals” and “other products” are used interchangeably and is used herein to include compounds suitable as liquid fuels, gaseous fuels, reagents, chemical feedstocks, chemical additives, processing aids, food additives, and other uses that chemicals can be put to, and includes, but is not limited to, hydrocarbons, hydrogen, methane, hydroxy compounds such as alcohols (e.g. ethanol, butanol, propanol, methanol, etc.), carbonyl compounds such as aldehydes and ketones (e.g. acetone, formaldehyde, 1-propanal, etc.), organic acids, derivatives of organic acids such as esters (e.g. wax esters, glycerides, etc.) and other functional compounds including, but not limited to, 1,2-propanediol, 1,3-propanediol, lactic acid, formic acid, acetic acid, succinic acid, pyruvic acid, enzymes such as cellulases, polysaccharases, lipases, proteases, ligninases, and hemicellulases and may be present as a pure compound, a mixture, or an impure or diluted form.

“Biocatalyst” is used herein to include enzymes and microorganisms, including solutions, suspensions, and mixtures of enzymes and microorganisms. In some contexts this word will refer to the possible use of either enzymes or microorganisms to serve a particular function, in other contexts the word will refer to the combined use of the two, and in other contexts the word will refer to only one of the two. The context of the phrase will indicate the meaning intended to one of skill in the art.

“Plant polysaccharide” is used herein to refer to polymers of sugars and sugar derivatives as well as derivatives of sugar polymers that occur in plant matter. Exemplary plant polysaccharides include lignin, cellulose, starch, and hemicellulose. Generally, the polysaccharide can have two or more sugar units or derivatives of sugar units. The sugar units and/or derivatives of sugar units may repeat in a regular pattern, or otherwise. The sugar units can be hexose units or pentose units, or combinations of these. The derivatives of sugar units can be sugar alcohols, sugar acids, amino sugars, etc.

“Biomass” is used herein to include biological material that can be converted into a biofuel, chemical or other product. One exemplary source of biomass is plant matter. Plant matter can be, for example, woody plant matter, non-woody plant matter, cellulosic material, lignocellulosic material, hemicellulosic material, carbohydrates, pectin, starch, inulin, fructans, glucans, corn, sugar cane, grasses, switchgrass, bamboo, and material derived from these. Plant matter can be further described by reference to the chemical species present, such as proteins, polysaccharides and oils. Polysaccharides include polymers of various monosaccharides and derivatives of monosaccharides including glucose, fructose, lactose, galacturonic acid, rhamnose, etc. Plant matter also includes agricultural waste byproducts or side streams such as pomace, corn steep liquor, corn steep solids, distillers grains, peels, pits, fermentation waste, straw, lumber, sewage, garbage and food leftovers. These materials can come from farms, forestry, industrial sources, households, etc. Another non-limiting example of biomass is animal matter, including, for example milk, meat, fat, animal processing waste, and animal waste. “Feedstock” is frequently used to refer to biomass being used for a process, such as those described herein.

“Fermentable sugars” is used herein to refer to sugars and/or sugar derivatives that can be utilized as a carbon source by the microorganism, including monomers, dimers, and polymers of these compounds including two or more of these compounds. In some cases, the organism may break down these polymers, such as by hydrolysis, prior to incorporating the broken down material. Exemplary fermentable sugars include, but are not limited to glucose, xylose, arabinose, galactose, mannose, rhamnose, cellobiose, lactose, sucrose, maltose, and fructose

“Broth” is used herein to refer to inoculated medium at any stage of growth, including the point immediately after inoculation and the period after any or all cellular activity has ceased and can include the material after post-fermentation processing. It includes the entire contents of the combination of soluble and insoluble matter, suspended matter, cells and medium, as appropriate.

“Saccharification” is used herein to refer to the conversion of plant polysaccharides to lower molecular weight species that can be utilized by the organism at hand. For some organisms, this would include, conversion to monosaccharides, disaccharides, trisaccharides, and oligosaccharides of up to about seven monomer units, as well as similar sized chains of sugar derivatives and combinations of sugars and sugar derivatives. For some organisms the allowable chain-link may be longer and for some organisms the allowable chain-link may be shorter.

“Pretreatment” or “pretreated” is used herein to refer to any mechanical, chemical, thermal, biochemical process or combination of these processes whether in a combined step or performed sequentially, that achieves removal or disruption of lignin so is to make the cellulose and hemicellulose polymers in the plant biomass more available to cellulolytic enzymes and/or microbes. In some embodiments, pretreatment can include removal or disruption of lignin so is to make the cellulose and hemicellulose polymers in the plant biomass more available to cellulolytic enzymes and/or microbes. In some embodiments, pretreatment can include the use of a microorganism of one type to render plant polysaccharides more accessible to microorganisms of another type. In some embodiments, pretreatment can also include disruption or expansion of cellulosic and/or hemicellulosic material. Steam explosion, and ammonia fiber expansion (or explosion) are well known thermal/chemical techniques. Hydrolysis, including methods that utilize acids and/or enzymes can be used. Other thermal, chemical, biochemical, enzymatic techniques can also be used.

### DESCRIPTION

The following description and examples illustrate certain preferred embodiments of the present invention in detail. Those of skill in the art will recognize that there are numerous variations and modifications that are encompassed by its scope. Accordingly, the description of a preferred embodiment should not be deemed to limit the scope of the present invention.

Various embodiments of the invention offer benefits relating to: 1) rendering cellulosic and hemicellulosic polymers of lignocellulosic material bioavailable, whether by making the polymers more accessible, hydrolyzing them, derivatizing, or acting on them in these or other ways which allow them to be utilized by the organism at hand, in a progressive manner throughout the process rather than rely on the effectiveness of a single initial pretreatment step, 2) utilizing multiple biocatalysts to achieve more complete saccharification of plant polymers, more complete conversion of plant-derived sugars to fuels and/or chemicals, more rapid conversion of plant-derived sugars to fuels and/or chemicals, 3) utilizing aerobic microorganisms to remove oxygen from the process to enable the subsequent use of anaerobic microorganisms, or utilizing anaerobic microorganism prior to the subsequent use of an anaerobic or aerobic microorganism 4) recycling nutrients within the process to minimize the cost of media, and 5) providing a method of making two or more products simultaneously in the process. Some embodiments of the invention may exhibit all of these benefits, while other embodiments may exhibit fewer, yet still remain within the scope of this disclosure.

In one preferred embodiment, a feedstock such as agricultural crops, crop residues, trees, woodchips, sawdust, paper, cardboard, and other materials containing cellulose, hemicellulose, and/or lignocellulose (collectively, “Feedstock”) either pretreated or not is contacted with an anaerobic organism capable of converting one or more of the plant polysaccharides in the feedstock to lower molecular weight specie(s) which can be utilized as a carbon source by a second microorganism in the production of fuel and/or other chemicals. These lower molecular weight species may remain as extracellular compounds, may be taken up as intracellular compounds, or be present as both intracellular and extracellular compounds. The organism may also at least partially polymerized or combined in some other way these compounds.

In one such embodiments, Clostridium phytofermentans is used. Clostridium phytofermentans includes American Type Culture Collection 700394T, and can in some embodiments be defined based on the phenotypic and genotypic characteristics of a cultured strain, ISDgT (Warnick et al., International Journal of Systematic and Evolutionary Microbiology, 52:1155-60, 2002). Aspects of the invention generally include systems, methods, and compositions for producing fuels, such as ethanol, and/or other useful organic products involving, for example, strain ISDgT and/or any other strain of the species Clostridium phytofermentans, including those which may be derived from strain ISDgT, or separately isolated. Some exemplary species can be defined using standard taxonomic considerations (Stackebrandt and Goebel, International Journal of Systematic Bacteriology, 44:846-9, 1994): Strains with 16S rRNA sequence homology values of 97% and higher as compared to the type strain (ISDgT), and strains with DNA re-association values of at least about 70% can be considered Clostridium phytofermentans. Considerable evidence exists to indicate that many microbes which have 70% or greater DNA re-association values also have at least 96% DNA sequence identity and share phenotypic traits defining a species. Analyses of the genome sequence of Clostridium phytofermentans strain ISDgT indicate the presence of large numbers of genes and genetic loci that are likely to be involved in mechanisms and pathways for plant polysaccharide fermentation, giving rise to the unusual fermentation properties of this microbe which can be found in all or nearly all strains of the species Clostridium phytofermentans. Clostridium phytofermentans strains can be natural isolates, or genetically modified strains.

Clostridium phytofermentans provides useful advantages for the conversion of biomass to ethanol and other products. One advantage of the Clostridium phytofermentans is its ability to produce enzymes capable of hydrolyzing polysaccharides and higher saccharides to lower molecular weight saccharides, oligosaccharides, disaccharides, and monosaccharides. In some embodiments, the organism can be used to hydrolyze various higher saccharides present in biomass to lower saccharides, such as in preparation for fermentation to produce ethanol, hydrogen, or other chemicals such as organic acids including formic acid, acetic acid, and lactic acid. Another advantage of the Clostridium phytofermentans is its ability to hydrolyze polysaccharides and higher saccharides that contain hexose sugar units, that contain pentose sugar units, and that contain both, into lower saccharides and in some cases monosaccharides. These enzymes and/or the hydrolysate can be used in fermentations to produce various products including fuels, and other chemicals. Another advantage of the Clostridium phytofermentans is its ability to produce ethanol, hydrogen, and other fuels or compounds such as organic acids including acetic acid, formic acid, and lactic acid from lower sugars such as monosaccharides. Another advantage of the Clostridium phytofermentans is its ability to perform the combined steps of hydrolyzing a higher molecular weight biomass containing sugars and/or higher saccharides or polysaccharides to lower sugars and fermenting these lower sugars into desirable products including ethanol, hydrogen, and other compounds such as organic acids including formic acid, acetic acid, and lactic acid.

Another advantage of the Clostridium phytofermentans is its ability to grow under conditions that include elevated ethanol concentration, high sugar concentration, low sugar concentration, utilize insoluble carbon sources, and/or operate under anaerobic conditions. These characteristics, in various combinations, can be used to achieve operation with long fermentation cycles and can be used in combination with batch fermentations, fed batch fermentations, self-seeding/partial harvest fermentations, and recycle of cells from the final fermentation as inoculum.

In some embodiments, Clostridium phytofermentans is contacted with pretreated or non-pretreated feedstock containing cellulosic, hemicellulosic, and/or lignocellulosic material. Additional nutrients can be present including nitrogen-containing compounds such as amino acids, proteins, hydrolyzed proteins, ammonia, urea, nitrate, nitrite, soy, soy derivatives, casein, casein derivatives, milk powder, milk derivatives, whey, yeast extract, hydrolyze yeast, autolyzed yeast, corn steep liquor, corn steep solids, monosodium glutamate, and/or other fermentation nitrogen sources, vitamins, and/or mineral supplements. In some embodiments, one or more additional lower molecular weight carbon sources can be added or be present such as glucose, sucrose, maltose, corn syrup, lactic acid, etc. Such lower molecular weight carbon sources can serve multiple functions including providing an initial carbon source at the start of the fermentation period, help build cell count, control the carbon/nitrogen ratio, remove excess nitrogen, or some other function.

The contacting with C. phytofermentans will generally include at least a period of time with sufficiently low dissolved oxygen to allow the organism to multiply and/or produce cellulolytic enzymes and/or store sugar/polysaccharide/oligosaccharides materials within the cell and/or produce fuel and/or other chemicals. Suitably low dissolved oxygen conditions can be achieved by any suitable method including heating the medium, purging the medium, broth, or fermenter with a low oxygen gas, addition of an anaerobic organism, exclusion of air during medium preparation, etc.

In some embodiments, Clostridium phytofermentans cells are cultured in an anaerobic environment, which can be achieved and/or maintained by bubbling a substantially oxygen-free gas through a bubbler that includes gas outlets that are submerged below a surface of the medium. Excess gas and effluent from reactions in the medium fill headspace, and are eventually vented through a gas outlet aperture formed in vessel wall. Gases that can be used to maintain anaerobic conditions include N₂, N₂/CO₂ (80:20), N_{0.2}/CO₂/H₂ (83:10:7), and Nobel gases, e.g., helium and argon. Methods to achieve anaerobic conditions are described in U.S. application Ser. No. 11/698,722 filed Jan. 26, 2007 entitled Systems and Methods for Producing Biofuels and Related Materials, incorporated herein by reference in its entirety.

In certain embodiments, the C. phytofermentans can reduce the molecular weight of one or more polysaccharides in the feedstock and incorporate at least a portion of the lower molecular weight compounds within the cell. After incorporation of these compounds, the cells are lysed and contacted with another organism for the production of fuel and/or other chemicals. The lysis can occur prior to, during, or after inoculation with the second organism. The relative timing of the lysis and the second inoculation can depend on such things as the method of lysis used, the robustness of the second organism, and the robustness of the C. phytofermentans. Under some circumstances, the broth may be inoculated with the second organism simultaneous to or prior to inoculation with C. phytofermentans. Organisms suitable for use as the second organism include those capable of producing ethanol, methanol, propanol, butanol, hydrogen, methane, lactic acid, acetic acid, succinic acid, pyruvic acid, formaldehyde, acetone or other compounds that would be fuels and/or other chemicals. Preferred organisms include yeasts, such as Saccharomyces cerevisiae, Clostridia species such as C. thermocellum, C. acetobutylicum, and C. cellovorans, and ethanol producing bacteria such as Zymomonas mobilis.

Suitable methods of lysis include, alone or in combination, addition of enzymes such as lysozyme, proteases, lipases, polysaccharases, addition of chelating agents such as phosphates, EDTA, carbonates, ion exchange resin, etc., high shear mixing, ultrasonic treatment, pressure-drop homogenization, addition of acids or bases, addition of oxidizing or reducing agents, or other suitable means.

The fuel and/or other compounds produced can be recovered by suitable processing methods depending on the particular material produced and the level of purity desired. For example, when producing ethanol the entire contents of the reaction can be transferred to a distillation unit, and 96 percent ethanol/4 percent water (by volume) can be distilled and collected. Fuel grade ethanol (99-100 percent ethanol) can be obtained by azeotropic distillation of the 96 percent ethanol, e.g., by the addition of benzene and then re-distilling the mixture, or by passing the 96 percent ethanol through molecular sieves to remove the water.

In one aspect the invention employs sequential aerobic or anaerobic cycling for the bioconversion of cellulosic/lignocellulosic material to fuels and chemicals.

Some embodiments employ aerobic/anaerobic cycling for the bioconversion of cellulosic/lignocellulosic material to fuels and chemicals. In some embodiments, the anaerobic microorganism can ferment biomass directly without the need of a pretreatment. In certain embodiments, feedstocks are contacted with biocatalysts capable of breaking down plant-derived polymeric material into lower molecular weight products that can subsequently be transformed by biocatalysts to fuels and/or other desirable chemicals.

Process steps in accordance with one embodiment may include: 1) contacting the feedstock with an aerobic cellulolytic microorganism, 2) contacting the resulting treated feedstock with an anaerobic cellulolytic microorganism that is also capable of fermenting sugars to fuels and/or chemicals, 3) separating a solids portion (including at least a portion of the microbial cells, residual feedstock and partially metabolized feedstock) from a liquid portion (including at least a portion of the fuels and/or other chemicals), 4) processing the solids by mechanical, thermal and/or chemical techniques to achieve at least partial breakdown of plant polymers in the residual feedstock material and to make available carbohydrate (e.g., monosaccharides, disaccharides, oligosaccharides, polysaccharides, sugar alcohols and other derivatives of sugar) and other nutrients associated with the cells of the microorganisms resulting from prior process steps, and 5) contacting the processed solids with a microorganism capable of transforming at least some of the carbohydrates present to fuels and/or other chemicals.

In some embodiments, the feedstock may be pretreated, such as by thermal, mechanical, and/or chemical means. Such pretreatment may at least partially hydrolyze carbohydrates or proteins present, disrupt cellular structure, increase the surface area, or render carbohydrates more accessible to microorganisms or enzymes.

In some embodiments, process steps include: 1) contacting a pre-treated biomass material under aerobic conditions with a first culture of an aerobic bacterium, where aerobic bacterium is capable of at least partially hydrolyzing the pre-treated biomass, 3) incubating the aerobic broth until the cellulolytic aerobic microorganism consumes at least a portion of the oxygen and hydrolyzes at least a portion of the biomass, thereby converting the aerobic broth into an anaerobic broth comprising a hydrolysate comprising fermentable sugars and 2) treating the with a second culture of a microorganism comprising an anaerobic microorganism capable of converting capable of converting fermentable sugar into biofuels.

Some embodiments employ anaerobic/aerobic cycling for the bioconversion of cellulosic/lignocellulosic material to fuels and chemicals. Other embodiments employ anaerobic/anaerobic cycling for the bioconversion of cellulosic/lignocellulosic material to fuels and chemicals. In some embodiments, the anaerobic microorganism can ferment biomass directly without the need of a pretreatment.

In some embodiments, process steps include: 1) contacting a biomass material under anaerobic conditions with a first culture of a non-genetically modified anaerobic bacterium, where the biomass has not been treated with exogenously supplied chemicals or enzymes, and where the non-genetically modified anaerobic bacterium converts at least a portion of the biomass into monosaccharides and disaccharides and 2) treating the biomass with a second culture of a microorganism that is not an obligate aerobe, wherein the monosaccharides and disaccharides are converted to a biofuel. The process can take place in a close container. In some embodiments the anaerobic bacterium is C. phytofermentans. In some embodiments, the second culture is Saccharomyces cerevisiae, Clostridia species such as C. thermocellum, C. acetobutylicum, and C. cellovorans, or Zymomonas mobilis. In some embodiments, the process steps can also include: 3) separating and recovering the resulting biofuel the residual biomass and cultures.

In some embodiments, the invention includes process for producing a biofuel comprising subjecting biomass which includes cellulose and hemi-cellulose containing plant materials to fermentation under mesophilic conditions in the presence of co-cultures of Clostridium phytofermentans and Zymonomas mobilis, the ratio of the cultures being in an amount whereby the conversion ratios of cellulose:ethanol and hemi-cellulose:ethanol are greater than the ratios obtained by use of either Clostridium phytofermentans or Zymonomas mobilis alone. In some embodiments, the conversion ratios obtained with co-cultures of Clostridium phytofermentans and Zymonomas mobilis are 5%, 10%, 15%, 20%, 25%, 30%, 35%, 40%, 45, 50%, 55%, 60%, 65%, 70%, 75%, 80%, 85%, 90%, 95%, or 100% greater than the ratios obtained by use of either Clostridium phytofermentans or Zymonomas mobilis alone. Mesophilic conditions are preferably maintained from about 28° to at about 35°.

In some embodiments, the invention provides for a process of producing a biofuel, comprising subjecting biomass which includes cellulose and hemi-cellulose containing plant materials to fermentation under mesophilic conditions in the presence of co-cultures of Clostridium phytofermentans and a second Clostridium species selected from the group consisting of Clostridium acetobutyliticum, Clostridium thermocellum, and Clostridium cellovorans, the ratio of the cultures being in an amount whereby the conversion ratios of cellulose:ethanol and hemi-cellulose:ethanol are greater than the ratios obtained by use of either Clostridium phytofermentans or the second Clostridium species alone. In some embodiments, the conversion ratios obtained with co-cultures of Clostridium phytofermentans and a second Clostridium species are 5%, 10%, 15%, 20%, 25%, 30%, 35%, 40%, 45, 50%, 55%, 60%, 65%, 70%, 75%, 80%, 85%, 90%, 95%, or 100% greater than the ratios obtained by use of either Clostridium phytofermentans or second Clostridium species alone.

In some embodiments, the invention provides for a process for simultaneous saccharification and fermentation of cellulosic solids from biomass into biofuel. The process comprised treating the biomass in a closed container with a Clostridium phytofermentans bacterium under conditions wherein the Clostridium phytofermentans bacterium produces saccharolytic enzymes sufficient to substantially convert the biomass into monosaccharides and disaccharides. The culture is then contacted with a culture of a second microorganism where the second organism is capable of substantially converting the monosaccharides and disaccharides into biofuel. Examples of second cultures include but are not limited to Saccharomyces cerevisiae, Clostridia species such as C. thermocellum, C. acetobutylicum, and C. cellovorans, and Zymomonas mobilis.

In some embodiments, the invention provides a process of producing a biofuel from a lignin-containing biomass. The process comprises: 1) contacting the lignin-containing biomass with an aqueous alkaline solution at a concentration sufficient to hydrolyze at least a portion of the lignin-containing biomass; 2) neutralizing the treated biomass to a pH between 7 to 8; 3) treating the biomass in a closed container with a Clostridium phytofermentans bacterium under conditions wherein the Clostridium phytofermentans bacterium produces saccharolytic enzymes sufficient to substantially convert the treated biomass into monosaccharides and disaccharides; and 4) introducing a culture of a second microorganism wherein the second organism is capable of substantially converting the monosaccharides and disaccharides into biofuel.

In some embodiments, the invention provides a process of producing a biofuel and nutrient fermentation residual from biomass. The process comprises: 1) treating biomass with a culture comprising Clostridium phytofermentans that, in a fermentation reaction, produces an alcohol and a fermentation residual comprising a nutrient selected from the group consisting of amino acids, cofactors, hormones, proteins, vitamins and lipids; 2) fermenting the culture under conditions suitable for production of the biofuel and under conditions suitable for production the nutrient; 3) separating the biofuel from the culture; and 4) recovering the fermentation residual comprising the nutrient.

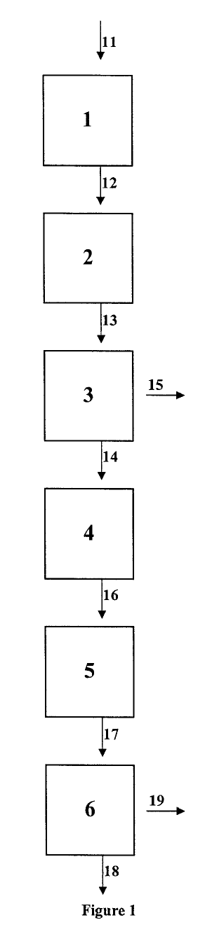

One embodiment of the invention is shown schematically in FIG. 1. The treated or untreated feedstock 11 is fed to an aerobic bioreactor 1 where it is acted upon by one or more aerobic microorganisms. Step 1 is an optional aerobic pretreatment step. The aerobically cultured feedstock 12 is then fed to an anaerobic bioreactor 2 where it is acted upon by one or more anaerobic microorganisms to produce one or more compounds useful as a fuel or other purposes, and optionally to depolymerize saccharides that are present. In another embodiment, the aerobic culturing 1 and anaerobic culturing 2 can be performed in the same vessel. The anaerobically treated material 13 is fed to a separator 3 where a substantially liquid portion 15 is separated from a solids-rich anaerobically treated residual portion 14. Step 3 is an optional separation step. Here, “substantially liquid portion” means the fraction resulting from the separation which has a lower suspended solids. This portion will generally be flowable and in some embodiments have a higher percent of light transmission then the other fraction. The substantially liquid portion 15 is further processed, as may be necessary, such as to isolate the fuel or desired chemicals. The separator 3 can be a density separation unit such as a settling tank, clarifier, centrifuge, hydrocyclone, etc., or it can be a membrane device such a reverse osmosis unit, crossflow microfiltration, ultrafiltration, nanofiltration, etc., or it can be a filtration unit or screening unit or flotation unit or a combination of these types of devices. The anaerobically treated residual portion 14 can be discarded, recycled, further processed or handled with a combination of these methods.

Further processing of the anaerobically treated residual portion 14 can include mechanical, chemical, and thermal treatment steps. In another embodiment, the residual portion 14 is processed by fermentation. In another embodiment, the residual portion 14 is processed with a combination of at least one treatment step and fermentation. The treated residual portion 14 can also be processed for the sale of one or more products present in it.

In FIG. 1, the anaerobically treated residual portion 14 is processed with a treatment step 4 to produce a treated residual material 16 that is then further processed in a fermentation step 5 to produce a fermented residual material 17. The fermented residual material 17 is then treated in a separation step 6 to partially or fully isolate useful products such as fuel or chemicals. In one embodiment, the separation step 6 separates a substantially liquid phase 19 from a solids-rich phase 18. The substantially liquid phase 19 can then be further processed to isolate and/or purify materials useful as fuel or chemicals. At least some portion of the substantially liquid phase 19 can also be recycled within the process. The solids-rich phase 18 can be discarded, recycled, landfill, composted, used to fertilize crops, or put to other purposes related to its composition.

Another embodiment of the invention provides a closed system process for the production of ethanol comprising the steps: 1) carrying out ethanol-producing anaerobic fermentation of biomass in a fermentation vessel at a temperature of at least about 35° C. in the presence of a Clostridium phytofermentans bacterium capable of producing sugars from biomass and in the presence of a facultatively anaerobic bacterium capable of fermenting sugars both aerobically and anaerobically and producing ethanol in anaerobic fermentation, 2) continuously withdrawing a portion of the fermentation medium from anaerobic fermentation; 3) separating bacteria from the withdrawn fermentation medium and recycling the separated bacteria to anaerobic fermentation; and 3) removing ethanol from the withdrawn portion of the fermentation medium. In one embodiment the facultatively anaerobic bacterium is E. Coli.

Another embodiment of the invention provides a process for producing ethanol from a cellulosic substrate comprising the steps of: 1) providing within a reaction vessel a reaction mixture in the form of a slurry comprising cellulosic substrate rendered anaerobic, saccharolytic enzymes, a Clostridium phytofermentans bacterium and optionally a Zymomonas mobilis bacterium; 2) agitating the reaction mixture for a first selected time interval, wherein the reaction mixture is reacted under conditions sufficient to initiate and maintain a fermentation reaction; 3) ceasing agitation of the reaction mixture for a sufficient period of time to permit insoluble substrate of the reaction mixture to settle during a second selected time interval, thereby forming an ethanol containing effluent layer substantially free of suspended solids and a residual solids layer; 4) removing from the reaction vessel the ethanol-containing effluent upon expiration of the second selected time interval, and before any further agitation; 5) adding a second reaction mixture, comprising the components of the reaction mixture of step 1), to the reaction vessel which contains the residual solids; and 6) repeating steps (2) through (5) to maintain a continuous fermentation reaction.

The invention further provides compositions. In some embodiments, the invention provides a composition comprising fermenting biomass containing a first culture of Zymomonas mobilis and a second culture of Clostridium phytofermentans in an amount not inhibiting Zymomonas growth and fermentation, in the substantial absence of exogenously supplied enzymes, air and added nutrients.

Feedstock—Cellulosic, Hemicellulosic and Lignocellulosic Material Sources.

The feedstock that may contain cellulosic, hemicellulosic, and/or lignocellulosic material may be derived from agricultural crops, crop residues, trees, woodchips, sawdust, paper, cardboard, grasses, and other sources.

Cellulose is a linear polymer of glucose where the glucose units are connected via β(1→4) linkages. Hemicellulose is a branched polymer of a number of sugar monomers including glucose, xylose, mannose, galactose, rhamnose and arabinose, and can have sugar acids such as mannuronic acid and galacturonic acid present as well. Lignin is a cross-linked, racemic macromolecule of mostly p-coumaryl alcohol, conferyl alcohol and sinapyl alcohol. These three polymers occur together in lignocellusic materials in plant biomass. The different characteristics of the three polymers can make hydrolysis of the combination difficult as each polymer tends to shield the others from enzymatic attack.

In some embodiments, the feedstock material can be subjected to optional mechanical, thermochemical, and/or biochemical pretreatment prior to being used in a bioprocess for the production of fuels and chemicals, but untreated lignocellulosic material can be used in the process as well. Mechanical processes can reduce the particle size of lignocellulosic material so that it can be more conveniently handled in the bioprocess and can increase the surface area of the feedstock to facilitate contact with chemicals/biochemicals/biocatalysts. The lignocellulosic material can also be subjected to thermal and/or chemical pretreatments to render plant polymers more accessible, but because various embodiments can incorporate multiple steps of lignocellulose treatment it may be possible to use milder and less expensive thermochemical pretreatment conditions. The addition of enzymes to break down plant polymers (saccharification) can be utilized as a component of conventional lignocellulosic bioconversion processes and these enzymes can constitute a significant cost.

Mechanical processes include, are not limited to, washing, soaking, milling, size reduction, screening, shearing, and size classification processes. Chemical processes include, but are not limited to, bleaching, oxidation, reduction, acid treatment, base treatment, sulfite treatment, acid sulfite treatment, basic sulfite treatment, and hydrolysis. Thermal processes include, but are not limited to, sterilization, steam explosion, holding at elevated temperatures in the presence or absence of water, and freezing. Biochemical processes include, but are not limited to, treatment with enzymes and treatment with microorganisms. Various enzymes that can be utilized include cellulases, amylase, β-glucosidase, xylanase, gluconase, and other polysaccharases; lysozyme; laccase, and other lignin-modifying enzymes; lipoxygenase, peroxidase, and other oxidative enzymes; proteases; and lipases. One or more of the mechanical, chemical, thermal and biochemical processes can be combined or used separately. Such combined processes can also include those used in the production of paper, cellulose products, microcrystalline cellulose, and cellulosics and can include pulping, kraft pulping, acidic sulfite processing. The feedstock can be a side stream or waste stream from a facility that utilizes one or more of these processes on a cellulosic, hemicellulosic or lignocellulosic material, such as a paper plant, cellulosic plant, cotton processing plant, or microcrystalline cellulose plant. The feedstock can also include cellulose-containing or cellulosic containing waste materials.

In preparation for inoculating the feedstock with the anaerobic organism, additional nutrients such as a nitrogen source, salts, vitamins, and trace elements can be added to the broth. Prior to inoculation, simultaneous to, or after inoculation, the oxygen level of the broth is reduced to a level suitable for the particular organism being used. One preferred organism is C. phytofermentans. Various means can be employed to reduce the oxygen level. For example, the broth or fermenter can be flushed with nitrogen or non-oxygen containing gas stream, the medium can be made up with oxygen being excluded, the medium can be heated, or an aerobic organism can be added. In one embodiment, an aerobic organism or facultative anaerobic organism that is also capable of making the desired fuel and/or other chemical is utilized. The transition between first organism to second organism can then be accomplished by changing the aeration pattern and selectively lysing the first organism.

Anaerobic Bioreactor

Of particular interest to the subject invention are anaerobic cellulolytic microorganisms that have the ability to break down cellulose and hemicellulose, and to metabolize both hexose and pentose sugars resulting from the saccharification of lignocellulosic material (for example Clostridium phytofermentans). To promote the growth of the anaerobic cellylolytic biocatalyst that can also transform sugars to fuels and/or chemicals it will be necessary to add nutrients to promote rapid growth and biochemical activity, however, the microbial culture chosen for use in the process can be selected, at least in part based on the simplicity and low cost of the nutrients it requires. While anaerobic microorganisms that can simultaneously saccharify lignocellulosic material and transform the full range of hexose and pentose sugars resulting from plant polymers into fuels and/or chemicals, the rate at which each type of hexose or pentose sugar is converted to fuels and/or chemicals will vary. Consequently, some sugars will be transformed by the anaerobic biocatalyst to fuels and/or chemicals more quickly than others. Therefore, one embodiment of the subject invention allows for a sufficient contact time between the lignocellulosic material and the anaerobic cellulolytic-fermenting biocatalyst to achieve substantially complete saccharification, but only partial conversion of sugars to fuels and/or products.

In one embodiment, an anaerobic culture including at least one anaerobe capable of hydrolyzing cellulose, hemicellulose, or lignocellulosic material and producing a desired fuel and/or other chemical is added to a portion of a feedstock, for example the feedstock 12. In another embodiment, an anaerobic culture including at least one anaerobe capable of hydrolyzing cellulose, hemicellulose, or lignocellulosic material and storing the hydrolyzed material intracellularly is added to a portion of the feedstock. In some embodiments the anaerobe added to the feedstock can both store hydrolyzed material intracellularly and produce a desired fuel and/or other chemical. In preferred embodiments, the anaerobe is C. phytofermentans.

Anaerobic cultures comprising C. phytofermentans are preferably maintained at about 30° for about 120 hrs. However, different organisms and different media compositions may require a temperature that is higher or lower and a fermentation time that is longer or shorter. During this time, the anaerobe can metabolize carbohydrate present in the broth to produce the desired fuel and/or other chemical and render the residual biomass more bioavailable for subsequent fermentation by another microbe. In other embodiments, the anaerobe hydrolyzes the carbohydrate present in the broth and stores the hydrolyzed material intracellularly. In other embodiments, the anaerobe hydrolyzes the carbohydrate and both stores a portion of the hydrolyzed material intracellularly and produces a fuel and/or other chemicals from a portion of the hydrolyzed and/or stored material. In some embodiments, such as when there is incompletely hydroyszed feedstock present, the anaerobe may further hydrolyze at least a portion of the remaining feedstock. The fuel and/or other chemicals produced will typically collect in the extracellular medium, however, in some instances, the fuel and/or other chemical will collect in another location. For example, gaseous products, including hydrogen, may build up in the head space of a bioreactor from which it can be vented, captured, etc. Other fuel and/or other chemical compounds may collect intracellularly.

Optional Separation of Anaerobically Treated Feedstock

The anaerobic fermentation step can be stopped when the feedstock is depleted, the hydrolysis of plant polysaccharides has slowed, the storage of carbon by the organism has slowed, or for some other reason such as to maintain a smooth fermentation plant operation. Various methods can be used to monitor the activity of the organism and to identify the point to stop the anaerobic fermentation including, but not limited to, monitoring of the off-gas rate and/or composition, broth pH, and medium composition. In some cases, the rate of CO₂ production and/or the rate of hydrogen production can be monitored, in the fermentation stopped when the production rate decreases. The broth can then be fractionated into a solids-rich portion and primarily liquid portion such as by centrifugation, settling, filtration, treatment with membranes, hydrocyclone, etc. For example, in FIG. 1 the broth can then be fractionated into a solids-rich portion 14 and a primarily liquid portion 15.

In various embodiments, the primarily liquid portion (e.g. liquid portion 15) can contain one or more desirable fuels and/or other chemical which can be further purified or used directly. Examples of products include alcohols, enzymes, organic acids and organic acid esters. The purification methods employed can include concentration methods such as evaporation, ultrafiltration, etc., crystallization, precipitation such as with salts or addition of a nonsolvent, liquid-liquid separation, distillation, chromatography, ion exchange, adsorption, dialysis and drying.

In some embodiments, one or more products would be contained in the solids-rich portion or a product can be in the solids-rich portion and the same or a different product can be in the primarily liquid portion. When a product is contained in the solids-rich portion, the solids-rich portion can be treated as the product itself, or this portion can be further processed, such as by drying, washing, lysing, extracting, derivitizing and/or by other techniques to achieve the desired purity and product characteristics.

Alternatively, the anaerobically treated feedstock may be directly separated into a product material, and a residual portion. An example of this approach would be the distillation of ethanol from the anaerobically treated feedstock with the still bottoms being the residual portion (e.g. residual portion 14).

A variation on this approach would be the recovery of one or more gaseous products, such as hydrogen or methane, during the course of the fermentation. In the embodiment depicted in FIG. 1, the separation 3 could occur during the fermentation within the fermentation vessel rather than after stopping the fermentation and would involve the separation of a gaseous product stream 15 from the fermentation broth 14.

Liquid Fraction

The primarily liquid portion (e.g. liquid portion 15) will frequently contain the fuels and/or chemicals produced during the anaerobic fermentation. Recovery of the desired fuel and/or other chemicals will depend on the specific compound produced by the microorganism. For example, in the case of alcohols, such as ethanol, methanol, propanol, butanol, etc., the liquid portion can be distilled to produce a high concentration alcohol, which can then be further dehydrated, such as with molecular sieves, pervaporation, additional distillation steps including those with an agent to break an azeotrope or otherwise facilitate the separation, or other techniques to perform the separation. The desired fuel and/or other chemicals can also be purified to remove other trace components, or it can be used as is.

Treatment of Anaerobically Treated Residual Portion and of the Anaerobically Treated Broth When the Optional Separation Step is not Utilized

An anaerobically treated residue (e.g. anaerobically treated residue 14) or anaerobically treated broth (e.g. anaerobically treated broth 13), when an optional separation step (e.g., separation step 3) is not utilized, is subjected to a mechanical, thermochemical, and/or biochemical treatment (e.g. biochemical treatment 4) to further release recalcitrant plant polymers, saccharify plant polymers, and/or release unmetabolized sugars or stored sugars/sugar polymers and soluble nutrients from the intracellular contents of microbial cells. The material so treated will be referred to as “treated residual material” whether or not the optional separation step (e.g. separation step 3) was used in the processing. The treated residual material (e.g. treated residual material 16) resulting from this further treatment of process solids or anaerobically treated broth may be used for animal feed, burned as fuel, or otherwise utilized, such as by additional processing or recycling within the process.

The processing techniques used may include cell lysis, such as by sonication, high shear mixing, steam explosion, treatment with enzymes, treatment with chemicals, osmotic shock, or other appropriate techniques. Other processing techniques may include hydrolysis of proteins and/or polysaccharides, such as by treatment with enzymes, acids, temperatures, or other appropriate techniques.

Other products, such as extracellular and/or secreted enzymes, can be recovered by appropriate techniques. Examples of such techniques can include ultrafiltration, nanofiltration, reverse osmosis, filtration, centrifugation, gravity settling, flotation, drying, dialysis, salt precipitation, precipitation by the addition of a nonsolvent, precipitation at or near the isoelectric point, as well as by combinations of these methods and other methods.

Enzymes that can be utilized include lysozyme, proteases, polysaccharases, lipases, alone or in combination. In some embodiments, more than one enzyme within one of these classes may be used, such as when two or more proteases are used. These can be utilized by the addition of the individual enzymes in purified or partially purified form, or by the addition of an enzyme cocktail with multiple enzymes and types of enzymes present.

In some embodiments, a chemical, thermal, or mechanical treatment can be utilized with the enzyme addition. Such additional treatment can be conducted before, during, and/or after the addition of the enzymes. Such treatments can include heating, cooling, changes in osmotic pressure, addition of chelating agents, high shear mixing, homogenization, sonication, addition of oxidizing agents, addition of reducing agents, and combinations of these as well as other appropriate techniques.

In some embodiments, the intracellular material exposed by the lysis step can include sugar-containing compounds. Hydrolysis of these sugar-containing compounds, including polysaccharides, may release monosaccharides, disaccharides, trisaccharides, or higher saccharides. Generally, the purpose of such hydrolysis would be to increase the bioavailability of these sugars for subsequent culturing with microorganisms. This culturing may be as part of a recycle step, or for a subsequent downstream fermentation step.

Fermentation Of Treated Residual Material

The treated residual material (e.g. residual material 16) can be fermented with one or more additional organisms, for example, an anaerobic microorganism, to produce fermented residual material (e.g. fermented residual material 17) which includes one or more compounds useful as a fuel or as a chemical. Exemplary additional microorganisms include S. cerevisiae, Z. mobilis, Clostridium acetobutylicum, C. phytofermentans, C. thermocellum, C. cellovorans, as well as other organisms that produce or are engineered to produce alcohols, organic acids, organic acid derivatives, aldehydes, ketones, hydrogen, or methane.

In some embodiments, the additional organism can be one that preferentially utilizes sugars that are only slowly utilized by the microorganism used in the anaerobic culture step. For example, for C. phytofermentans, slowly utilized sugars include lactose, arabinose and xylose, while more rapidly utilized sugars include glucose, cellobiose, and galactose. With Z. mobilis, more rapidly utilized sugars include glucose, fructose, and sucrose. The product generated at this step may be the same or different from that produced in the anaerobic fermentation step. Fermentation conditions for the treated residual material will vary according to the specific organism being used and the final product desired. For C. phytofermentans, a temperature of about 28 to about 38° C. or preferably about 33 to 36° C. or about 35° C. is used under conditions to exclude oxygen and at a pH of less than about 8.5. Other culture conditions can be found in U.S. patent application Ser. No. 11/698,727, entitled System and Methods for Producing Biofuels and Related Materials, filed Aug. 2, 2007, and in Thomas A. Warrick et al., Clostridium phytofermentans sp. nov., A Cellulolytic Mesophile from Forest Soil, 52 International Journal of Systematic and Evolutionary Microbiology 1155 (2002); incorporated in their entirety herein by reference thereto.

Separation of the Fermented Residual Material

The fermented residual material (e.g. fermented residual material 17) can be separated into a solids-rich portion (e.g. solids-rich portion 18) and a primarily liquid portion (e.g. liquid portion 19) such as by centrifugation, settling, filtration, treatment with membranes, hydrocyclone, etc.

In some embodiments, the fermented residual material may be directly separated into a product material, and a solids-rich material. An example of this approach would be the distillation of ethanol from the anaerobically treated feedstock with the still bottoms being the solids-rich material (e.g. solids-rich material 18). In other embodiments, such as when the product is a gas, such as hydrogen or methane, the separation of the product from the fermented residual material (e.g. fermented residual material 17) can occur in the fermenter by the removal of the gas phase from the fermenter. Both when the product is separated directly from the broth and when a gaseous product is collected from the fermenter, additional purification or treatment steps can be performed on the product stream.

Solids-Rich Material

The solids-rich portion (e.g. solids-rich portion 18) generally contains lignocellulosic material and microbial cells. In some embodiments, this fraction may be discarded, used as animal feed, used as fertilizer, or landfilled. In other embodiments, the solids may be processed with mechanical, chemical, thermal methods, or combinations of these. The treated solids can have additional products recovered, or they can be recycled to an upstream point in the process, or they can, for example, be processed in an additional fermentation step. In some embodiments, the solids rich material maybe treated to isolate a primarily liquid portion containing sugars and/or nutrients. This primarily liquid portion may be utilized in the same fashion or in a different fashion from the rest of the material.

Subsequent Fermentation of Process Solids

In various embodiments, the separated solids-rich fractions are recycled to an earlier culturing step to allow more complete conversion of plant polymers to sugars and useful products than would otherwise be possible. In some embodiments, milder treatments and/or less expensive treatment steps may be possible as compared to “once-through” processes because the lignocellulosic material will be significantly softened as the result of microbial action on the Feedstock. Furthermore, the recycling of cellular material liberated from the cells grown in the cultured stages of the process can serve as nutrients for those stages which can result in a cost reduction.

In other embodiments, the treated process solids are cultured with an additional organism to produce a fuel and/or other chemicals. Preferred organisms include those that rapidly utilize the sugars that are only slowly utilized by the microorganism used in the anaerobic culture step. The product generated at this step may be the same or different from that produced in the anaerobic fermentation step.

Optional Aerobic Bioreactor Pretreatment

In some embodiments, lignocellulosic material, pretreated or not, can be optionally contacted with live aerobic cellulolytic microorganisms (for example Trichoderma reesei) that will simultaneously and/or sequentially promote saccharification and consume oxygen. The in situ production of saccharification enzymes can reduce process costs and the removal of oxygen creates an environment suitable for the growth of anaerobic cellulolytic microorganisms.

In one embodiment, a culture containing at least one aerobic cellulolytic microorganism is added to the feedstock. Additional nutrients, such as a nitrogen source, vitamins, minerals and trace elements can be added as needed by the microorganism. Sufficient inoculum is added to provide good growth within a reasonable time. The pH can be controlled or buffered to a suitable range for the microorganism. Aeration can be provided as needed for the organism and the temperature operated within a suitable range.

In another embodiment, a culture containing at least one aerobic cellulolytic microorganism, as above, is added to a slurry containing the feedstock and nutrients sufficient for the aerobic cells to proliferate. This culture is maintained at an appropriate temperature and pH for the organism with sufficient aeration to support growth of the organism. In addition, an oxygen enriched gas stream may be used in place of or in combination with the air stream to achieve the desired cell activity or dissolved oxygen level.

At the end of the aerobic fermentation, as determined by monitoring the rate of hydrolysis of lignocellulosic substrate or other appropriate techniques, the aeration is reduced to allow the organism to consume the remaining oxygen in preparation for growth of an anaerobic culture. Various means can be employed to reduce the oxygen level. For example, in a batch culture, the inlet air can simply be turned off, or the air can be replaced with nitrogen or an oxygen depleted stream. Alternatively, the culture can be transferred (e.g., via pipe) to another vessel and during the transfer, the culture is cut off from an oxygen supply. In a continuous culture, the broth may go through a zone where oxygen is not added. Such zone may be an area of the bioreactor, a pipe, another vessel, etc.

### EXAMPLES

### Example 1

### Preparation of MB1 Media

MB1 media can be prepared by mixing in a beaker:
a. 750 mL double distilled H₂O
b. 8 g of K₂HPO₄
c. 4 g of KH₂PO₄
d. 1 g of (NH₄)₂SO₄
e. 6 g of cysteine
f 6 g of Amberex695AG 6 g (inexpensive yeast extract: may use Bacto)
g. substrate—for example, cellobiose, glucose, cellulose, as well as other substrates described herein

If substrate is insoluble, (e.g. corn stover, paper sludge) the pH of the media is adjusted without pH without substrate. The media is distributed into separated containers and the substrate is added separately to each container. The standard for inoculum propagation is 0.3% cellobiose. The pH is adjusted to 7.50 with NaOH or KOH. ddH₂O is added to bring to a volume of 1 L. The media is distributed into separate containers, sealed and made anaerobic.

The media is autoclaved for a minimum of 20 minutes on liquid setting @121° C. The time is increase time if the total volume in autoclave is greater than 3 L or the containers hold more than 500 mL. Other times and temperatures may be used, with the limitation that too much heat or for too long may disrupt the sugar substrate. Amberex 695AG can be obtained from Sensient Flavors Co., 330 S. Mill Street, Juneau Wis. 53039 (920-386-4527).

Substrate can be added before autoclaving. Resazurin does not need to be used. Optionally, Resazurin can be added to assure that container is anaerobic. The ingredients are all placed into a beaker. Water is added and pH adjusted if necessary to pH 7.50 with 6N KOH. The mix is heated in microwave to just boiling. After prompt removal, the Hungate method is used to make anaerobic or the vacuum manifold is used to make the test tubes or flasks anaerobic with nitrogen gas.

If the vacuum manifold is used then media should be placed in test tubes or flasks/bottles that can accommodate rubber septa. Flanged test tubes are typically sealed with rubber septa (such as Cat #2048-11800 Bellco Glass Inc, Vineland N.J.) or for larger culture volumes screw cap bottles/flasks are used equipped with an appropriately sized rubber septa held in place with a screw cap. The rubber septa of test tubes and flasks can subsequently be pierced with sterile needles to add inoculum or substrate, or to remove samples for analysis.

### Example 2

### Preparation of GS-2 Media

GS-2 media can be prepared by mixing in a beaker:
a. 750 mL double distilled H₂O
b. 2.90 g of K₂HPO₄
c. 1.50 g of KH₂PO₄
d. 2.10 g of Urea
e. 2.00 g of cysteine HCl
f. 10.00 g of MOPS
g. 3.00 g of sodium citrate tribasic*2H₂O
h. 6.00 g of Bacto yeast extract (Catalogue #212750 Becton, Dickinson Co.)
i. 1.00 mL of 0.1% resazurin
j. substrate—for example, cellobiose, glucose, cellulose, as well as other substrates described herein
   ddH₂O is added to make up 900 mL. If substrate is insoluble, (corn stover, paper sludge) the pH is adjusted without substrate. Then the media is distributed into separate container, and the substrate is added separately to each container. Standard for inoculum propagation is 0.3% cellobiose. The pH is adjusted to 7.50 with NaOH or KOH.

The media is transferred to a round bottom flask and heat to boiling in the microwave, without boiling over the media. The flask is placed on a heated stir plate near a Hungate apparatus and kept heated to just below boiling while flushing with N₂ gas until the medium “depinks”

8.7 ml are distributed per tube under a constant N₂ atmosphere, flush with N₂ until resazurin is colorless. The media is cooled to room temperature, then, cooled in an ice bath for 10 minutes.

The tubes are placed in an autoclave press with the top plate and pad firmly screwed down onto the tops of the stoppers. (Test tube racks are used with rubber pads at the base to prevent tube cracking.)

The media is autoclaved for a minimum of 20 minutes on liquid setting @121° C. The time is increased if the total volume in autoclave is greater than 3 L or the containers hold more than 500 mL. Other times and temperatures may be used, with the limitation that too much heat or for too long may disrupt the sugar substrate. After autoclaving the tubes are allowed to come to room temperature and remove from the autoclave press.

Using the Hungate apparatus and a constant stream of N2 gas, 1.0 ml of sterile GS-2 salts are added. 10% v/V GS-2 salts are added after autoclaving. The recipe for GS-2 salts is described below:

GS-2 Salts

| | | |
|---|---|---|
| a. ddH₂O | 100 | mL |
| b. MgCl₂*6H₂0 | 1.0 | g |
| c. CaCl₂*2H₂0 | 0.15 | g |
| d. FeSO₄*7H₂0 | 0.00125 | g |

The media may be made anaerobic or used aerobically. If aerobic, the media will turn a little pink when added; it should de-pink shortly due to cysteine in the media. Autoclave as for media (above).

After both are cool and before inoculating, 10% v/V salts are added to media: if tubes of media, for a final volume of 10 mL, 9 mL GS-2 media and 1 mL GS-2 salts.

### Example 3

### Media Autoclaving Procedure

Autoclaving

The temperature is set to 121° C. which equals a pressure of 15 psi.

Liquid cycle: if total volume media in autoclave is less than or equal to 3 liters, run for 20 min if total volume media in autoclave is greater than 3 L or if individual containers hold 500 mL or more, increase autoclave time.

The biggest limiting factor for time and temperature of autoclave cycle is that there is a danger of the soluble sugar in the media “carmelizing”—it turns a dark brown and the texture changes. This changes the properties of the sugar and is to be avoided.

Containers

| | | |
|---|---|---|
| 10 | mL media vol | Bellco Glass anaerobic tubes, 20 mm mouth |
| | | stopper with Bellco blue 20 mm stoppers |
| | | stopper with Wheaton grey 20 mm stoppers |
| | | crimp with 20 mm aluminum crimps, from Bellco or Wheaton |
| | | crimps secured with “crimper” tool from Bellco or Wheaton |
| | | Hungate tubes |
| | | tube is special order only for ~$8/tube |
| | | stoppers are green rubber, tapered to fit, available from various places |
| 50 | mL media vol | Serum bottles, 20 mm mouth |
| | | stopper with Bellco blue 20 mm stoppers |
| | | stopper with Wheaton grey 20 mm stoppers |
| | | crimp with 20 mm aluminum crimps, from Bellco or Wheaton |
| | | crimps secured with “crimper” tool from Bellco or Wheaton |
| 100-200 | mL media vol | Graduated 250 mL serum bottles, 30 mm mouth |
| | | stopper with Wheaton grey 30 mm stoppers |
| | | crimp with 30 mm aluminum crimps, from Wheaton |
| | | crimps secured with “crimper” tool from Wheaton |
| <200 | mL media vol | Graduated 250 mL screw-top media bottles |
| | | stoppered with EDPM Lyophilization-Style black stoppers |
| | | secured with plastic screw-cap w/center hole |
| | | Graduated 500 mL, 1 L or 2 L screw-top media bottles |
| | | stoppered with EDPM Lyophilization-Style black stoppers |
| | | secured with plastic screw-cap w/center hole |

### Example 4

### Procedure for Removal of Oxygen from Media

Anaerobic indicator: resazurin−pink=aerobic
colorless=anaerobic

Resazurin should be colorless by the time the media goes into the autoclave. If it is pink, there is oxygen present in the container.

However, cysteine is a slow reducer and it is not unusual to take 10 minutes for a 100 mL bottle to de-pink after gassing.

If the media is still pink when it comes out of the autoclave, or turns pink after shaking a container fresh from the autoclave, oxygen is present.

When gassing MBI: if the media is too dark to see resazurin color reliably, then a container is prepared with Anaerobic Indicator Solution equal to the volume of containers of distributed media. The recipe for Anaerobic Indicator Solution is described below

Anaerobic Indicator Solution 100 mL
a. 100 mL ddH₂O
b. 0.06 g cysteine
c. 0.01 mL 1% resazurin solution
   Container is gassed of Anaerobic Indicator Solution simultaneously with media and use clearly visible resazurin reaction to judge media aerobic conditions.

Vacuum Manifold Procedure

This is a multipurpose device designed for use in preparing anaerobic environments in crimp top tubes and flasks, filtration of HPLC buffers, residual substrate filtration and vacuum drying.

Turning on the Nitrogen Tanks
a. Check to see that the nitrogen tank is properly connected to the regulator.
   i. IF CLOSED: relieve the pressure on the diaphragm by loosening Brass T-valve on the front of the regulator then open the valve on top of the nitrogen tank
   ii. Adjust regulator pressure to 3 or 4 psi with the large black dial
   iii. Turn the small brass dial on side of the regulator to allow nitrogen to flow to the N2/Vacuum selection valve

Activating the Cold Trap and Vacuum Pump
iv. Move the top portion of the cold trap w/the immersion chiller probe into the trap bucket, turn on the chiller.
v. Wait 10 minutes for the probe to cool the trap; you will notice the antifreeze freezing to the lower coils.
vi. Turn on the vacuum pump by pressing the switch on the power strip beside chiller.
b. Degassing Tubes and Flasks
   i. Apply 22 gauge needles to each of the five ports on the vacuum manifold. You may reuse needles.
   ii. Close all five black plastic valves on the main manifold. Do Not adjust the sixth black plastic valve attached to the over pressure release valve.
   iii. Pierce the septa of your tubes or flasks iv. Using the glass double oblique valve select the vacuum function
   v. Open the 5 black plastic manifolds valves and apply the vacuum to each port.
   vi. Vacuum tubes for 2 min and flasks with <500 mL for 2 min, flasks with 500 mL+for 5 min, at 300 mBars or less
   vii. Turn the glass selection valve to Nitrogen and flush tubes until the white Teflon ball at the bottom of the over pressure relief valve jingles. (About 20 seconds)
   viii. Check to see that the surface of the liquid stops rippling (about 5 more seconds)
   ix. Turn the glass double oblique valve to repeat vacuum cycle
   x. After 3^{rd} flush with Nitrogen, remove needles from septa, close all 5 ports, turn double oblique selector to vacuum and switch to 5 new tubes or flasks.
c. Filtering HPLC buffers
   i. Close all five black plastic valves on the main manifold. Do not adjust the sixth black plastic valve attached to the over pressure release valve.
   ii. Attach the side arm Erlenmeyer filtration flask to the large metal manifold via the 1-hole stopper. Seal the remaining ports with #8 stoppers.
   iii. Place a 0.45 um filter in the filter holder, secure with clamp and place in the Erlenmeyer flask
   iv. Using the glass valves open the vacuum line to the manifold.
   v. Pour buffer into the filter funnel and repeat until solution is filtered
   vi. Close the glass valve to the vacuum line turn of the vacuum pump
   vii. Open a manifold port with the black dial to restore to Atm pressure
   viii. Remove the filtered buffer.
d. Filtering residual Substrates
   i. Close all five black plastic valves on the main manifold. Do not adjust the sixth black plastic valve attached to the over pressure release valve.
   ii. Place a glass filtration funnel in each of the 6 ports on the manifold.
   iii. Place a pre-weighed 0.45 um filter in each funnel
   iv. Make sure the liquid trap for the filtration manifold is empty.
   v. Pour supernatants into the filter funnels
   vi. Turn glass valve to apply vacuum line to the manifold.
   vii. When filtration is complete close glass valves to stop vacuum flow. Restore to Atm pressure by opening a black plastic vlave
   viii. Remove the filter papers and residual solids
e. Activating the Vacuum Oven
   i. Close all five black plastic valves on the main manifold. Do not adjust the sixth black plastic valve attached to the over pressure release valve.
   ii. Attach the drying oven vacuum line to the manifold via the 1-hole stopper. Seal the remaining ports with #8 stoppers.
   iii. Turn the glass valves to apply vacuum line to the drying oven.
   iv. Use the dials on top of the oven to regulate the pressure. Seal valves when desired pressure is reached.
   v. When complete close glass valves to stop vacuum flow

Shut Down
f. Close glass valves to stop vacuum flow through the manifold.
g. Turn off vacuum pump.
h. Turn off chiller.
i. Turn off Nitrogen flow by the small brass dial on the side of the regulator. You may leave the tank open.
j. Restore the manifold to atmospheric pressure by opening a black plastic valve on the front of the manifold

Hungate Procedure

To Turn Hungate on
a. Slide glass tube so that all of the copper is inside the heating unit.
b. Turn on the rheostat to 120 volts by flipping the switch down: the dial on top regulates the heat—once adjusted, DO NOT move!
c. Turn on N₂ i. The large knob perpendicular to the displays should be loose
   ii. Open the main valve on the tank
   iii. Tighten the large knob until the needle in the dial on the left moves. It doesn't have to move much. This knob regulates the spring on the diaphragm which regulates the flow; they can be damaged by sudden changes in pressure, which is why it should be loose when opening the tank.
   iv. Adjust the flow by using the small side knob (in line with the displays) on the regulator. Allow a small flow through only one port until ready to gas media; this ensures positive flow of N₂ against atmosphere.
d. Allow copper to heat with air flow for 40 minutes before using the Hungate.

Using Hungate to Gas Out Media
a. Transfer media to a flask and heat to boiling in a microwave oven or on a hot plate—do not boil over.
b. Place the flask on a heated stir plate near the Hungate apparatus. Keep the flask heated just below boiling and flush with N2 from Hungate until the media de-pinks. Continue flushing and heating while distributing.
c. Distribute media into containers, each flushing with N2. Place stoppers lightly over flushing cannula.
*Media will pink up a little during distribution: flush until it de-pinks, while cooling in an ice bath to slightly less than room temperature.*
d. Remove cannula while holding stopper down to minimize atmospheric contamination.
e. Place tube rack in tube rack press. Secure stoppers for other containers. Autoclave as is appropriate to container and total volume (described above).

To Turn Hungate Off
a. All but one port should be closed, with a very small flow through that port.
b. Turn the rheostat off: the switch should be in the middle position (up=140v).
c. Slide glass tube so that all of the copper is out of the heating element.
d. Allow copper to cool 15-20 minutes (until it is comfortable to touch barehanded) before turning off the N₂.
e. Turn off the N₂ i. Close main tank valve
   ii. Wait for both dial needles to rest at zero.
   iii. When both dials read zero, release the diaphragm regulator by turning the large know counter-clockwise until loose

### Example 5

### Fermentation Conditions—C. phytofermentans

Temperature: 35° C. or 30° C.
a. 35° promotes growth and is used with soluble substrates
b. 30° used for insoluble substrates

Agitation: Test tube cultures are grown stationary, with no agitation

Cultures in flasks can be grown statically or at various agitation speeds. Agitation is useful if the substrate is insoluble (i.e., Avicel) and accessible substrate surface area might be a limiting factor. The speed of agitation is adjusted to keep the substrate in suspension and this is variable depending upon the substrate type and substrate concentration.

pH: 7.50 (see media SOP)

C. phytofermentans can grow from pH 6.5-8.5, best in the higher midrange

Substrate: 0.3% cellobiose—standard for inoculum maintenance
higher than 2.5% may cause inhibition

Some evidence that growth on glucose inhibits production of cellulose enzymes—this inhibition may be reversible with intermediary transfer to cellobiose before transfer to cellulose

### Transfer Conditions

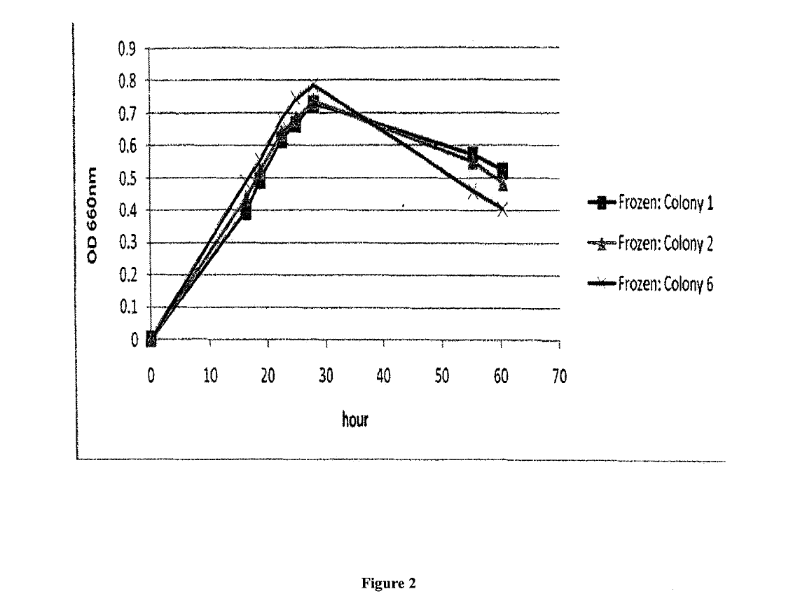

Typically the working stock of C. phytofermentans is maintained as an actively growing vegetative culture in GS-2 or MB1 media containing 0.3% cellobiose. Cultures are transferred in mid-log phase of growth (see below) using a 2% inoculum volume for propagation in test tubes. Bioreactors are typically inoculated using an inoculum volume of 10%. The volume of inoculum can be adjusted to achieve mid-log phase of growth at times that are needed to support the requirements of experiments. The growth of C. phytofermentans can be determined by measuring OD at a wavelength of 660 nm (FIG. 2). The Mid-log OD is substrate dependent:
a. 0.3% substrate: transfer at OD660 nm 0.500-0.700 absorbance units, substrate is limiting and culture will enter stationary phase shortly after 0.700 absorbance units
b. 1%+ substrate: transfer at OD 0.500-0.700 absorbance units

### Example 6

### Preparation of Frozen Stocks of C. phytofermentans

To prepare a frozen stock of C. phytofermentans, a mid-log phase culture is prepared, an equal volume of sterile glycerol (30% stock solution) is added, and placed in the −80° C. freezer. These frozen stocks can be stored indefinitely. To reactivate a frozen stock, the stock is thawed on ice and either the culture is streaked onto an appropriate agar plate or a 2% inoculum is used into liquid media, typically in a test tube. If the culture is streaked onto agar then 3 to 5 days of incubation anaerobically at 30° C. are allowed to obtain good growth. A sterile inoculation loop is used to transfer a single colony to one ml of sterile media (GS-2 or MB1) in a microfuge tube and agitate to produce a suspension of bacterial cells. A sterile syringe is used to draw up the bacterial suspension and to inoculate it into a sealed anaerobic test tube. If the thawed culture is to be added directly to a test tube, then a sterile syringe is used to draw up an appropriate volume (2% inoculum volume is typical) of microbial cell suspension and inject it through the septum of a sealed anaerobic test tube containing GS-2 or MB-1 media. All these operations are performed in an anaerobic glove box. After reactivating a frozen culture at least two subculture events are allowed before using as a vegetative working stock. This allows the frozen stock to acclimate to growth in liquid culture and yield reliable growth kinetics.

### Example 7

### Sugar and Ethanol Analysis

The data for product formation and remaining substrate concentration comes from HPLC analysis.

1) The culture is anaerobically sampled: 22 gauge needles are preferred for anaerobic sampling through stoppers. The following steps are followed
a. draw anaerobic gas into the syringe and expel it at least twice
b. draw up an amount of anaerobic gas equal to the sample volume into the syringe
c. inject the anaerobic gas into the container
d. take the sample (typically one to 1.5 ml)
   2) Sample is placed in microfuge tube and centrifuge for 10 min at 12000 rpm.
   3) Supernatant is filtered with a 0.45 micron syringe filter into HPLC sampling vial (may use insert).
   4) The samples are analyzed by high pressure liquid chromatography using an Aminex HPX-87H column operated at 55° C. with a 0.005 mM H₂SO₄ mobile phase. The concentration of cellobiose can also be quantified on the same column, or using an Aminex HPX-87P column at 80° C. with a water mobile phase.

### Example 8

### Evaluation of Culture for Contamination

Contamination can be detected in a variety of ways. For example:
a. By looking a sample under a microscope. If anything else other than C. phytofermentans is seen, then the sample is contaminated.
b. By monitoring the pH of the culture, contamination is suspected if the pH is below 6.8.
c. HPLC data can sometimes indicate one of the more common contaminants; if a very large amount of lactic acid is detected, the culture is contaminated.
d. A sample of the culture can be streaked onto agar plates (See SOP for agar medium to get recipe for C. phytofermentans selective agar or other agars). If morphologically distinct colonies grow up within the streak path, the culture is contaminated.

Examples of reagents that can be used in the Examples described herein and the ordering details are described in the table below.

Ordering Details

| | |
|---|---|
| Anaerobic tubes | Bellco 2048-00150 |
| blue 20 mm stoppers | Bellco 2048-11800 |
| grey 20 mm stoppers | VWR 16171-650 |
| 20 mm aluminum crimps | Bellco 2048-11020/VWR 16171-829 or VWR 16171-851 (tear- |
| | away) |
| 20 mm crimper | Bellco 2048-10020/VWR HP9301-07 |
| Serum bottles | VWR 16171-385 |
| Hungate tubes w/stoppers | Special order item |
| Hungate autoclave press | Special order item |
| Graduated 250 mL serum bottles 30 mm mouth | VWR 16171-420 |
| grey 30 mm stoppers | VWR 16171-630 |
| 30 mm aluminum crimps | VWR 16171-862 |
| 30 mm crimper | VWR 26676-406 |
| 250 mL screw-top media bottle | VWR 89000-236 (other size bottles with this type opening |
| | available) |
| black rubber EDPM stoppers | VWR 16171-620 |
| cap w/hole for use w/syringe | VWR 16149-145 |
| K₂HPO₄ | Sigma-Aldrich P3786 |
| KH₂PO₄ | Sigma-Aldrich P5379 |
| (NH₄)₂SO₄ | Sigma-Aldrich A2939 |
| cysteine HCl*H₂0 | Sigma-Aldrich C7880 |
| Amberex yeast extract | Sensient 695AG |
| D-(+)-cellobiose | MP Biomedicals 101298 |
| D-glucose, anhydrous | Mallinckrodt Chemicals 4912-12 |
| urea | Sigma-Aldrich U1250 |
| MOPS | Sigma-Aldrich M1252 |
| sodium citrate tribasic*2H₂0 | Sigma-Aldrich S4641 |
| Bacto yeast extract | BD 212750 |
| resazurin | Sigma-Aldrich R7017 |
| LB broth | VWR 900003-118 |
| Bacto-Agar | Difco 0140-01 |
| MgCl₂*6H₂0 | Sigma-Aldrich M2670 |
| CaCl₂*2H₂0 | Sigma-Aldrich 223506 |
| FeSO₄*7H₂0 | Sigma-Aldrich 215422 |

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A process for making ethanol and other chemicals, comprising:
providing a pretreated biomass-derived material comprising a plant polysaccharide;
inoculating the pretreated biomass-derived material with a first culture comprising a cellulolytic aerobic microorganism in the presence of oxygen to generate an aerobic broth, wherein the aerobic microorganism is capable of at least partially hydrolyzing the plant polysaccharide;
incubating the aerobic broth until the cellulolytic aerobic microorganism consumes at least a portion of the oxygen and hydrolyzes at least a portion of the plant polysaccharide, thereby converting the aerobic broth into an anaerobic broth comprising a hydrolysate comprising fermentable sugars;
inoculating the anaerobic broth with a second culture comprising an anaerobic microorganism capable of converting fermentable sugars into ethanol and other chemicals; and
fermenting the inoculated anaerobic broth until at least a portion of the fermentable sugars have been converted into ethanol and other chemicals.

2. The process of claim 1, wherein at least a portion of the ethanol is recovered from the fermented anaerobic broth.

3. The process of claim 1 or 2, further comprising lysing the aerobic and anaerobic microorganisms in the fermented anaerobic broth to produce a lysate comprising remaining fermentable sugars and cellular contents.

4. The process of claim 3, further comprising subjecting the lysate to additional physical and/or chemical treatment.

5. The process of claim 3, further comprising inoculating the lysate with another microorganism and/or enzyme cocktail capable of accelerating the conversion of the remaining fermentable sugars into ethanol and other chemicals.

6. A process for making a material suitable as a fuel, comprising:
providing a plant-derived material comprising a polysaccharide;
inoculating the plant-derived material with a first culture comprising a cellulolytic aerobic microorganism to generate a broth, wherein the aerobic microorganism is capable of at least partially hydrolyzing the polysaccharide;
incubating the broth in the presence of oxygen such that at least a portion of the polysaccharide is hydrolyzed to one or more sugar species;
incubating the broth under conditions to reduce the oxygen concentration so as to convert the broth to an anaerobic broth;
inoculating the anaerobic broth with a second culture comprising an anaerobic microorganism capable of converting the one or more sugar species into a material suitable as a fuel;
fermenting the inoculated anaerobic broth to produce a material suitable as a fuel.

7. The process of claim 6, wherein at least a portion of the material suitable as a fuel is recovered from the fermented anaerobic broth.

8. The process of claim 6 or 7, further comprising lysing the aerobic and anaerobic microorganisms in the fermented anaerobic broth to produce a lysate comprising intracellular sugars and cellular contents.

9. The process of claim 8, further comprising subjecting the lysate to additional physical and/or chemical treatment.

10. The process of claim 9, further comprising inoculating the lysate with another microorganism.

11. The process of claim 6, wherein the plant-derived material is pretreated prior to the inoculation with a cellulolytic aerobic microorganism.

12. The process of claim 6, wherein the material suitable as a fuel comprises ethanol.

13. A process for making ethanol and other chemicals, comprising:
providing a biomass-derived material comprising a plant polysaccharide;
incubating the biomass-derived material with a first culture comprising cell of Clostridium phytofermentans under anaerobic conditions to hydrolyze at least a portion of the plant polysaccharide wherein the cells of the culture incorporate at least a portion of the hydrolyzed plant polysaccharide as an intracellular compound;
lysing the cells of Clostridium phytofermentans to produce a lysed broth;
inoculating the lysed broth with a second culture comprising an anaerobic microorganism capable of converting fermentable sugars into ethanol and/or other chemicals; and
fermenting the inoculated anaerobic broth until at least a portion of the fermentable sugars have been converted into ethanol and/or other chemicals.

14. A method of producing a biofuel, comprising the steps of:
providing a biomass material under anaerobic conditions in a closed container, wherein the biomass has not been treated with exogenously supplied chemicals or enzymes;
treating the biomass with a first culture of a non-genetically modified anaerobic bacterium, wherein the non-genetically modified anaerobic bacterium converts at least a portion of the biomass into monosaccharides and disaccharides; and
treating the biomass with a second culture of a microorganism that is not an obligate aerobe, wherein the monosaccharides and disaccharides are converted to a biofuel.

15. The method of claim 14 wherein said first culture of non-genetically modified bacterium is Clostridium phytofermentans.

16. The method of claim 14 wherein said second culture of a microorganism is selected from the group consisting of S. cerevisiae, Z. mobilis, Clostridium acetobutylicum, C. phytofermentans, C. thermocellum, C. cellovorans.

17. The process of claim 14, further comprising lysing the aerobic microorganisms in the fermented anaerobic broth to produce a lysate comprising remaining fermentable sugars and cellular contents.

18. The process of claim 17, further comprising subjecting the lysate to additional physical and/or chemical treatment.

19. The method of claim 14 further comprising separating and recovering the converted biofuel from the residual biomass and cultures.

20. The method of claim 14 wherein said biomass includes cellulose and hemi-cellulose containing materials.

21. The method of claim 14 wherein said biomass includes lignin.

22. The method of claim 22 further comprising contacting said biomass with aqueous alkaline solution at a concentration sufficient to hydrolyze at least a portion of the lignin-containing biomass and neutralizing the treated biomass to a pH between 7 to 8.

23. A method of producing a biofuel, comprising subjecting biomass which includes cellulose and hemi-cellulose containing plant materials to fermentation under mesophilic conditions in the presence of co-cultures of Clostridium phytofermentans and a second Clostridium species selected from the group consisting of Clostridium acetobutyliticum, Clostridium thermocellum, and Clostridium cellovorans, the ratio of the cultures being in an amount whereby the conversion ratios of cellulose:ethanol and hemi-cellulose:ethanol are greater than the ratios obtained by use of either Clostridium phytofermentans or the second Clostridium species alone.
